SEQUENCE LISTING (2 sequences)

SEQ ID NO: 1 - DNA, 4654 bp, Homo sapiens
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaataaaaaaaaaaaaaaa

SEQ ID NO: 2 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

(19) United States
(12) Patent Application Publication
(10) Pub. No.: US 2012/0289416 A1
(43) Pub. Date: Nov. 15, 2012

(54) METHODS AND KITS USED IN ASSESSING CANCER RISK

(75) Inventors: Pamela Pollock, Queensland (AU); Paul J. Goodfellow, St. Louis, MO (US)

(73) Assignees: WASHINGTON UNIVERSITY, St. Louis, MO (US); THE TRANSLATIONAL GENOMICS RESEARCH INSTITUTE, Phoenix, AZ (US)

(21) Appl. No.: 13/384,567

(22) PCT Filed: Jul. 19, 2010
(86) PCT No.: PCT/US10/42400

§ 371 Date: Apr. 6, 2012

Related U.S. Application Data
(60) Provisional application No. 61/226,666, filed on Jul. 17, 2009.

(51) Int. Cl. C12Q 1/68; H01J 49/26; C40B 30/00; G01N 30/00; G01N 33/566; G01N 33/53

(52) U.S. Cl. 506/7; 435/7.23; 436/501; 435/6.12; 73/61.52; 250/282

(57) ABSTRACT

Methods of assessing the risk of recurrence of endometrial cancer on the basis of the presence or absence of mutations in FGFR2 are disclosed.

### BACKGROUND OF THE INVENTION

Endometrial cancer is the most common gynecological cancer. Endometrial carcinoma is subdivided into Type I and Type II disease. Type I endometrioid endometrial accounts for approximately 80-85% of endometrial cancers and is classified as being estrogen-dependent and well differentiated. Type II endometrial cancers comprise poorly differentiated endometrioid, clear cell, and papillary serous histological subtypes that display high biological aggressiveness and are associated with poor prognosis. Approximately 75% of type I endometrioid tumors are diagnosed as Stage I/II. These patients have a 5 year overall survival of 80-90%, a 5 year cancer specific survival of 90-95% and a recurrence rate of 4-8% (Creutzberg et al. 2000). However, for those women that recur, or present with advanced stage or progressive disease, survival is poor as there are no adjuvant therapies proven to be effective. The median survival after recurrence is 10 months and the 5-year survival for patients who have recurred is only 13%. There is a clear need to develop additional prognostic markers to identify those patients at risk for recurrence.

### BRIEF SUMMARY OF THE INVENTION

The present invention provides among other things a method of assessing the risk of disease recurrence in patients diagnosed with endometrial cancer.

It is an object of the invention to classify a subject into a cohort of increased risk of recurrence of endometrial cancer based upon FGFR2 mutation status.

It is an object of the invention to provide a kit used to classify a subject into a cohort of increased risk of recurrence of endometrial cancer based upon FGFR2 mutation status.

It is an object of the invention to identify endometrial cancer patients with a higher risk of recurrence of disease that would be otherwise predicted based on existing clinico-pathological risk factors such as stage, grade, age, or race among others.

The above and other objects may be achieved through the use of methods involving obtaining a sample from the subject and subjecting the sample to conditions that allow detection of a mutant of either SEQ ID NO. 1 or SEQ ID NO. 2. The subject is known to have had endometrial cancer and the sample comprises a tumor cell. The cohort comprises two or more individuals with an increased risk of recurrence of endometrial cancer. The mutant may comprise any mutation in SEQ ID NO. 1 or SEQ ID NO. 2, including those that lead to one or more the following amino acid changes: S252W, P253R, S373C, Y376C, C383R, G385R, I548V, N550K, N550H, K660E, M392R, V396D, L398M, and IVS 10+2A>C. The endometrial cancer may be of the endometrioid subtype. The stage may be any stage, including Stage IA, Stage IB, Stage IC, Stage IIA, and Stage IIB. The grade may be any grade, including Grade 1, Grade 2, and Grade 3. The conditions may allow detection of a mutant of SEQ ID NO. 1. In this example, the conditions may comprise the use of a technology selected from the group consisting of nucleic acid sequencing, microarray analysis, PCR amplification, allele specific PCR amplification, restriction fragment length polymorphism, allele specific hybridization, allele specific primer extension, and/or Southern Blot. The conditions may comprise detection of a mutant of SEQ ID NO. 2. In this example, then the conditions may comprise use of a technology selected from the group consisting of HPLC, mass spectrometry, ELISA, flow cytometry, immunohistochemistry or radioimmunoassay. The conditions may alternatively comprise measuring the activity of FGFR2 protein.

The above and other objects may be achieved through the use of kits comprising a first reagent capable of detecting a mutant of a sequence selected from the group consisting of SEQ ID NO. 1 and SEQ ID NO. 2 and an indication of a result that signifies classification of a subject into a cohort where the cohort comprises two or more individuals with an increased risk of recurrence of endometrial cancer. The mutant may comprise any mutation in SEQ ID NO. 1 or SEQ ID NO. 2, including those that lead to one or more the following amino acid changes: S252W, P253R, S373C, Y376C, C383R, G385R, I548V, N550K, N550H, K660E, M392R, V396D, L398M, and IVS 10+2A>C. The first reagent may be capable of binding to a mutant of SEQ ID NO. 1. In that example, the kit may further comprise a component that facilitates the use of a technology selected from the group consisting of nucleic acid sequencing, microarray analysis, PCR amplification, allele specific PCR amplification, restriction fragment length polymorphism, allele specific hybridization, allele specific primer extension, and Southern blot. The kit may comprise a first reagent that is capable of binding a mutant of SEQ ID NO. 2. The first reagent may comprise a first antibody. In this example, the kit may further comprise a component that facilitates the use of a technology selected from the group consisting of ELISA, flow cytometry, and radioimmunoassay. The result may be any result that signifies the detection of a mutant including a particular nucleic acid sequence or optical density value. The indication may be any indication including a positive control or a writing. A writing may be any writing including a writing on paper or a writing made available via a website. A writing may comprise a photograph. The indication may also comprise software configured to detect the result as input and the classification of the subject as output. Such software may be incorporated into a machine configured to detect the mutant.

### BRIEF DESCRIPTION OF THE FIGURES

FIG. 1 depicts the location of mutations identified in FGFR2 in endometrioid endometrial cancers. The majority of the mutations occur at seven hotspots.

FIG. 2 depicts progression free survival curves in intermediate risk patients with (Yes) and without (No) an FGFR2 mutation.

FIG. 3 depicts overall survival in intermediate patients with (Yes) and without (No) an FGFR2 mutation.

Elements and acts in the figures are illustrated for simplicity and have not necessarily been rendered according to any particular sequence or embodiment.

### DETAILED DESCRIPTION OF THE INVENTION

Endometrial cancer includes all forms and subtypes of the disease, including for example, serous, mucinous, and endometrioid histological subtypes or any other cancer that starts in the endometrium, which includes the lining of the uterus. Endometrial cancer is currently surgically staged using the International Federation of Gynecology and Obstetrics (FIGO) system, which emphasizes complete surgico-pathologic assessment of data. In response to the dismal prognosis associated with progressive or recurrent endometrial cancer, multiple efforts have been made to identify patients at risk for disease progression and recurrence. Patients who present with advanced extrauterine disease (stage III/IV) at diagnosis have a high risk of recurrence. In those patients that present with cancer confined to the uterus (Stage I/II), an increased risk of recurrence is associated with histologic cell type, tumor grade, depth of myometrial invasion, occult extension into the cervix and tumor cell invasion of lymphatic vessels (lymphovascular space invasion: LVSI). Table 4 demonstrates the stage and grade classifications of patients considered to have a low, intermediate or high risk of recurrence, where the intermediate risk is further broken down to patients with a low-intermediate risk and high-intermediate risk.

The concept of the FGFR2 gene encompasses a gene of human origin with a coding nucleotide sequence set forth in SEQ ID NO 1, or homologs including allelic variants and orthologs. The FGFR2 protein encompasses a protein, also preferably of human origin, having the amino acid sequence set forth in SEQ ID NO 2 or homologs, including orthologs thereof.

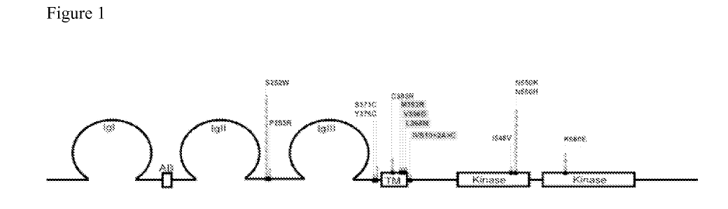

FIG. 1 displays the various domains of the FGFR2 protein and the FGFR2 mutations mapped in relation with the domains. FGFR2 belongs to a family of structurally related tyrosine kinase receptors (FGFRs 1-4) encoded by four different genes. FGFR2 is a glycoprotein composed of three extra-cellular immunoglobulin-like (1 g) domains, a transmembrane domain, and a split tyrosine kinase domain. Alternative splicing in the IgIII domain is primary determinant of both the patterns of redundancy and specificity in FGF/FGFR binding and signaling. This splicing event is tissue specific and gives rise to the IIIb and IIIc receptor isoforms for FGFRI-FGFR3 which possess distinct ligand specificities (Mohammadi M, Olsen S K and Ibrahimi O A. (2005), Cytokine Growth Factor Rev 16: 107-137, Ornitz D M and Itoh N. (2001). Genome Bioi 2: REVIEWS3005). For FGFR2, cells of an epithelial linage only express the “IIIb” isoform encoded by exon 8 (FGFR2b; SEQ ill NO:2; NP_{—}07529.2), while mesenchymally derived cells exclusively express the “IIIc” isoform utilizing exon 9 (FGFR2c; SEQ ill NO:3; NP_{—}000132.1) (Scotet E and Houssaint E. (1995). Biochim Biophys Acta 1264: 238-242). The FGFR2b iosform predominantly binds FGF1, FGF3, FGF7 and FGF10, while FGFR2c does not bind FGF7 and FGF10 but does bind FGF1, FGF2, FGF4, FGF6,and FGF8 with high affinity (Ibrahimi O A, Zhang F, Eliseenkova A V, Itoh N, Linhardt R J and Mohammadi M. (2004), Hum Mol Genet 13: 2313-2324).

An FGFR2 mutation with increased activity in a test subject or a biological sample may also be called an activation mutation. Activation mutations display higher total FGFR2 activity in the test subject or biological sample in comparison with a control, e.g., a healthy subject or a standard sample. Preferably, although not necessarily, the activity is at least 10%, at least 50%, at least 100%, or at least 150% higher in the test subject or sample than in the control. The increased activity, for example, may result from increased basal FGFR2 activity in the absence of ligand, increased level of activation in the presence of ligand, prolonged stimulation, delayed degradation or over-expression, e.g., due to enhanced ligand binding, promiscuous or inappropriate ligand binding, constitutive receptor dimerization, impaired recycling resulting in augmentation of signaling, delayed degradation, or kinase activation.

A higher expression level of FGFR2 may result from, for example, a mutation in a non-coding region of a FGFR2 gene or a mutation in a coding or non-coding gene involved in FGFR2 transcription or translation. The expression level of FGFR2 can be determined, for example, by comparing FGFR2 mRNA or the level of FGFR2 protein in a test subject as compared to a control, for example by comparing the tumor to normal endometrium (e.g., a normal adjacent endometrium sample).

Conserved variants encompass any mutation or other variant in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Depending on the location of the mutation in the overall context of the protein, the substitution may have little or no effect on the apparent molecular weight or isoelectric point of the protein or polypeptide. A conserved variant can still result in receptor activation by a wide variety of mechanisms.

Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. The concept of a variant further encompasses a polypeptide or enzyme which has at least 60%, 75%, 85%, 90%, or 95%, amino acid identity as determined by algorithms such as BLAST or FASTA and which has the same or substantially similar properties and/or activities as the native or parent protein or enzyme to which it is compared.

One example of such a variant is a gain-of-function variant. Gain of function variants of polypeptides encompass any variant in which a change in one or more amino acid residues in a protein or enzyme improves the activity of the polypeptide. Examples of activities of a polypeptide that may be improved by a change resulting in a gain of function variant include but are not limited to enzymatic activity, binding affinity, phosphorylation or dephosphorylation efficiency, activation, deactivation, or any other activity or property of a protein that may be quantitatively measured by some method now known or yet to be disclosed.

Proteins that possess a common evolutionary origin may be homologous or similar to one another. Examples of homologous or similar proteins include proteins from superfamilies (e.g., the immunoglobulin superfamily) and homologous proteins from different species. Such proteins and their encoding genes have sequence homology with one another. The homology may be expressed in terms of percent similarity or the presence of specific residues or motifs at conserved positions.

A mutation may be any detectable change in genetic material such as DNA, or a corresponding change in the RNA or protein product of that genetic material. A mutant may be any biological material in which one or more mutations are detected when compared to a control material. Examples of mutations include gene mutations, in which the DNA sequence of a gene or any controlling elements surrounding the gene is altered. Controlling elements include promoter, enhancer, suppressor or silencing elements capable of controlling a given gene. Other examples of mutations include alterations in the products of DNA expression such as RNA or protein that result from corresponding mutations in the DNA. Mutants may also be interchangeably called variants. The concept of a mutant includes any change in DNA sequence specific to the tumor cell (not present in DNA prepared from normal, non-neoplastic tissues).

Assessing the risk of a particular disease outcome includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a disease outcome also encompasses the concept of prognosis. A prognosis may be any assessment of the risk of disease outcome in an individual in which a particular disease has been diagnosed.

A sample may be any cell source from which DNA, including genomic, somatic, and germline DNA may be obtained. In endometrial cancer, a biological sample is often obtained from the uterus and generally includes one or more endometrial tumor cells. Circulating tumor cells may be found and obtained from serum. Tumor cells may be obtained by any method now known in the art or yet to be disclosed, including for example, surgical resection, laser capture microdissection, isolation from blood or other fluids including lavage fluid, or any other method capable of obtaining and, if necessary, concentrating endometrial tumor cells. Alternatively a sample may comprise free DNA from a tumor extracted directly from serum (See Reference 32).

A subject includes any human or non-human mammal, including for example: a primate, cow, horse, pig, sheep, goat, dog, cat, or rodent, capable of developing endometrial cancer including human patients that are suspected of having endometrial cancer, that have been diagnosed with endometrial cancer, or that have a family history of endometrial cancer. Methods of identifying subjects suspected of having endometrial cancer include but are not limited to: physical examination, family medical history, subject medical history, endometrial biopsy, or a number of imaging technologies such as ultrasonography, computed tomography, magnetic resonance imaging, magnetic resonance spectroscopy, or positron emission tomography. Methods of diagnosing endometrial cancer as well as the staging, grading, or other clinical delineation of endometrial cancer are well known to those of skill in the medical arts.

Sequence-specific oligonucleotides include sets of oligonucleotides that can be used to detect specific variations or mutations in the FGFR2 gene. Probes include oliognucleotides capable of forming a hybrid structure with a sequence in a target region due to complementarity of at least one nucleic acid base in the probe with a sequence in the target protein of the subject.

Prognostic methods encompass detecting a mutation in the FGFR2 protein including mutations that result in increased activity of the FGFR2 protein. Examples of such mutations include mutations occurring in the junction between the immunoglobulin-like (Ig) domains II and III; mutations occurring in the IgIII domain; mutations occurring in the junction between the IgIII domain and the transmembrane (TM) domain; mutations occurring in the TM domain; mutations occurring in the junction between the TM domain and the tyrosine kinase domain I; mutations occurring in the tyrosine kinase domain I, or mutations occurring in the tyrosine kinase domain II. Such mutations likely induce an amino acid substitution. Examples of such amino acid substitutions induced by mutations include but are not limited to: an S to W mutation at position 252, a P to R mutation at position 253, an S to C mutation at position 373, a Y to C mutation at position 376, a C to R mutation at position 383, an M to R mutation at position 392, a V to D mutation at position 396, an L to M mutation at position 398, an I to V mutation at position 548, an N to K mutation at position 550, an N to H mutation at position 550, and a K to E mutation at position 660 with position numbers as indicated in SEQ ID NO. 2. In one nonlimiting embodiment, the mutation is consist of a deletion of nucleotide C and T at position 2290-91 of the nucleotide sequence (NM-02297.2) or an IVS10+2A>C splicing mutation with position numbers as indicated in SEQ ID. NO. 1 or any other somatic mutation found in an endometrial tumor cell.

A detected FGFR2 receptor activation mutation may increase activation of the receptor by, for example, enhancing ligand binding, promoting altered or promiscuous ligand affinity with reduced selectivity, constitutive receptor dimerization, delayed degradation, impaired recycling from the cell membrane, signaling inappropriately from intracellular membranes, overexpression, or kinase activation.

In one embodiment, the prognosis of endometrial cancer in a subject may be assessed by determining an activity level of the FGFR2 protein in an endometrial cancer cell of a test subject and comparing it to the activity in endometrial cells of a control subject, wherein an increased activity of FGFR2 protein in the test subject compared to the control subject is indicative of an increased risk of recurrence of endometrial cancer. The level of FGFR2 activity may be assessed by determining the level of activity in a FGFR2 signaling pathway through any method now known or yet to be developed. Examples include but need not be limited to, assessing the expression of targets up- or down-regulated upon FGFR2 signaling, assessing the phosphorylation status of proteins phosphorylated or dephosphorylated on FGFR2 signaling, or any other method capable of detecting an increase in FGFR2 activity or ligand promiscuity.

Mutated forms of FGFR2 nucleic acids, such as in FGFR2 DNA or any transcripts (including any splice variants now known or yet to be disclosed) as well as a deregulated expression (including overexpression or underexpression) of FGFR2 or other elements of a FGFR2 pathway may be detected by any of a variety of suitable methods.

Any method capable of detecting a mutated nucleic acid in a biological sample now known or yet to be disclosed may be employed and many strategies of genotypic analysis are now known to those skilled in the art. Some of these methods use nucleic acid sequences such as specific oligonucleotides to detect mutations in an FGFR2 nucleic acid in a biological sample. Such oligonucleotides may specifically hybridize to a nucleic acid sequence containing the specific mutation, or to a region adjacent to the site of mutation. Other methods use primers that permit amplification of all or part of an FGFR2 nucleic acid. Alternatively, or in combination with such techniques, oligonucleotide sequencing described herein or known to the skilled artisan may be applied to detect the FGFR2 mutations. One skilled in the art may use hybridization probes in solution and in embodiments employing solid-phase procedures. In such procedures, the test nucleic acid is adsorbed or otherwise affixed to a selected matrix or surface. The fixed, single-stranded nucleic acid is then subjected to specific hybridization with selected probes. Alternatively, one skilled in the art may use oligonucleotide primers in an amplification technique, such as PCR or reverse-PCR (“reverse polymerase chain reaction”), to specifically amplify a target DNA or mRNA, respectively. Such primers include primers that permit amplification of FGFR2 exons.

One example of such a method includes but is not limited to the following: contacting a biological sample containing DNA with specific oligonucleotides permitting the amplification of all or part of the FGFR2 gene, the DNA contained in the sample having being rendered accessible, where appropriate, to hybridization, and under conditions permitting a hybridization of the primers with the DNA contained in the biological sample; amplifying said DNA; detecting the amplification products; and comparing the amplified products as obtained to the amplified products obtained with a normal control biological sample, and thereby detecting an abnormality in the FGFR2 gene if such abnormality is present and not detecting an abnormality if such abnormality is not present.

Alternatively, a sample may be sequenced directly with no amplification. In such methods, the sequenced DNA is compared to a normal genomic control sequence. The control sequence may be obtained from another subject or from a noncancerous sample from the same subject. One such method of sequencing is allele specific primer extension in which sample DNA hybridized to a chip is used as a synthesis template with the affixed oligonucleotide as a primer. Only the added dNTP's are labeled. Incorporation of the labeled dNTP then serves as a signal indicating the presence of the mutation. The fluorescent label may be detected by any of a number of instruments configured to read at least four different fluorescent labels on a DNA chip. In an alternative method, the identity of the final dNTP added to the oligonucleotide may be assessed by mass spectrometry. In this method, the dNTP's may, but need not be labeled with a label of known molecular weight.

Other methods of detecting abnormalities in FGFR2 include those that detect abnormalities in the transcript of the FGFR2 gene. Such methods include amplifying mRNA transcripts in a biological sample by techniques such as RT-PCR. One example of such a method includes but is not limited to the following: producing cDNA from mRNA contained in a biological sample; contacting said cDNA with specific oligonucleotides capable of amplifying of all or part of the transcript of the FGFR2 gene, under conditions capable of hybridizing the primers with said cDNA; amplifying said cDNA; detecting the amplification products; comparing the amplified products as obtained to the amplified products obtained with a normal control biological sample, and thereby detecting an abnormality in the transcript of the FGFR2 gene if such an abnormality is present and not detecting an abnormality if such an abnormality is not present. A control may be any noncancerous endometrial tissue control sample known as noncancerous to those skilled in the art, for example, a normal adjacent endometrium sample or a normal FGFR2 mRNA or DNA, obtained from blood, buccal swab or other source.

Samples to be used in mRNA analysis may be obtained from any cell source, as described above, including a biopsy tissue. RNA may be then isolated from the sample using standard methods well known to those of ordinary skill in the art. Examples include but are not limited to: guanidium thiocyanate-phenolchloroform extraction (Chomocyznski et al., Anal. Biochem., 1987, 162:156), isolation through the use of resin, Trizol® or other reagents, or any other appropriate method. The isolated RNA is then subjected to coupled reverse transcription and amplification by polymerase chain reaction (RT-PCR), using specific oligonucleotide primers that are specific for a selected region of the cDNA sequence. Primer annealing conditions are chosen to ensure specific reverse transcription and amplification; thus, the appearance of an amplification product is diagnostic of the presence of a particular genetic variation. In another embodiment, RNA is reverse-transcribed and amplified. Mutations in the amplified sequences (if present) may then be detected by any of a number of methods including direct sequencing, restriction fragment length polymorphism, hybridization of a specific probe to the amplified sequence, or be cloning into a plasmid followed by sequencing. If mutations are not present, then they will not be detected.

Nucleic acids that hybridize to mutant forms of FGFR2 may be used as probes in prognostic assays such a probe may comprise a substantially purified oligonucleotide that further includes a region having a nucleotide sequence that is capable of hybridizing specifically to a region of a FGFR2 gene that may be mutant or polymorphic. Such probes can then be used to detect specifically which, if any, mutation of the FGFR2 gene is present in a sample taken from a subject. The mutant or polymorphic region can be located in the promoter, exon, or intron sequences of the FGFR2 gene. In general, such probes have a sufficient number of nucleotides to allow specific hybridization to the target nucleotide sequence. Probes complementary to mutant sequences with the appropriate specificity may be constructed by those skilled in the art. For example, a portion of the FGFR2 gene may first be amplified and isolated from chromosomal DNA and hybridized to a probe. In such a case a probe of 10, 15, 20, 30, 50, or 100 nucleotides may be used.

The probe or primer may include a label. A label may be any substance capable of aiding a machine, detector, sensor, device, or enhanced or unenhanced human eye from differentiating a sequence that contains a particular allele from a cell that does not contain the allele. Examples of labels include but are not limited to: a radioactive isotope or chelate thereof, a dye (fluorescent or nonfluorescent,) stain, enzyme, or nonradioactive metal. Specific examples include but are not limited to: fluorescein, biotin, digoxigenin, alkaline phosphatase, biotin, streptavidin, ³H, ¹⁴C, ³²P, ³⁵S, or any other compound capable of emitting radiation, rhodamine, 4-(4′-dimethylamino-phenylazo)benzoic acid (“Dabcyl”); 4-(4′-dimethylamino-phenylazo)sulfonic acid (sulfonyl chloride) (“Dabsyl”); 5-((2-aminoethyl)-amino)-naphtalene-1-sulfonic acid (“EDANS”); Psoralene derivatives, haptens, cyanines, acridines, fluorescent rhodol derivatives, cholesterol derivatives; ethylenediaminetetraaceticacid (“EDTA”) and derivatives thereof or any other compound that signals the presence of bound ligand to an allele. In one embodiment of the invention, the label includes one or more dyes optimized for use in genotyping. Examples of such dyes include but are not limited to: dR110, 5-FAM, 6FAM, dR6G, JOE, HEX, VIC, TET, dTAMRA, TAMRA, NED, dROX, PET, and LIZ.

Alternatively, the probe may be modified to be more stable. Exemplary nucleic acid molecules that may be used to modify the probe to increase stability include phosphoramidate, phosphothioate and methylphosphonate analogs of DNA (see also U.S. Pat. Nos. 5,176,996; 5,264,564; and 5,256,775).

One may use HPLC or denaturing HPLC (DHPLC) techniques to analyze the FGFR2 nucleic acids. DHPLC was developed when observing that, when HPLC analyses are carried out at a partially denaturing temperature, homoduplexes can be separated from heteroduplexes having the same base pair length (Hayward-Lester, et al., Genome Research, 1995,5:494; Underhill, et al., Proc. Natl. Acad. Sci. USA, 1996, 93:193; Doris, et al., DHPLC Workshop, 1997, Stanford University). Thus, the use of DHPLC was applied to mutation detection (Underhill, et al., Genome Research,1997, 7:996; Liu, et al., Nucleic Acid Res., 1998, 26; 1396). DHPLC can separate heteroduplexes that differ by as little as one base pair. “Matched Ion Polynucleotide Chromatography” (MIPC), or Denaturing “Matched Ion Polynucleotide Chromatography” (DMIPC) as described in U.S. Pat. Nos. 6,287,822 or 6,024,878, are additional separation methods.

Alternatively, one can use the DGGE method (Denaturing Gradient Gel Electrophoresis), or the SSCP method (Single Strand Conformation Polymorphism) for detecting an abnormality in the FGFR2 gene. DGGE is a method for resolving multiple DNA fragments of identical length on the basis of sequence differences as small as a single base pair change, using electrophoresis through a gel containing varying concentrations of denaturant (Guldberg et al., Nuc. Acids Res. 1994, 22:880). SSCP is a method for detecting sequence differences between two DNAs, comprising hybridization of the two species with subsequent mismatch detection by gel electrophoresis (Ravnik-Glavac et al., Hum. Mol. Genet. 1994, 3:801). “HOT cleavage”, a method for detecting sequence differences between two DNAs, comprising hybridization of the two species with subsequent mismatch detection by chemical cleavage (Cotton, et al, Proc. Natl. Acad. Sci. USA 1988, 85:4397), can also be used.

Additionally, RT-PCR allows visualization of the consequences of a splicing mutation such as exon skipping or aberrant splicing due to the activation of a cryptic site.

Techniques using microarrays including microarrays that utilize high-throughput screening, may also be advantageously implemented to detect genetic abnormalities or assess gene expression. Gene expression may be that of the FGFR2 gene or the expression of another gene upstream or downstream in a pathway of which FGFR2 is a component or any other gene the expression of which correlates with FGFR2 expression. Microarrays may be designed so that the same set of identical oligonucleotides is attached to at least two selected discrete regions of the array, so that one can easily compare a normal sample, contacted with one of said selected regions of the array, against a test sample, contacted with another of said selected regions. These arrays use microfluidic conduits to avoid the mixture of normal sample and test sample. Examples of microarray techniques include those developed by Nanogen, Inc (San Diego, Calif.) and those developed by Affymetrix. However, all types of microarrays, also called “gene chips” or “DNA chips”, may be adapted for the identification of mutations. Such microarrays are well known in the art.

The solid support on which oligonucleotides are attached may be made from glass, silicon, plastic (e.g., polypropylene, nylon), polyacrylamide, nitrocellulose, or other materials now known or yet to be disclosed. One method for attaching the nucleic acids to a surface is by printing on glass plates, as is described generally by Schena et al., Science 1995, 270:467-470. This method is especially useful for preparing microarrays of cDNA. See also DeRisi et al., Nature Genetics 1996, 14:457-460; Shalon et al., Genome Res. 1996, 6:639645; and Schena et al., Proc. Natl. Acad. Sci. USA 1995,93:10539-11286.

Other methods for making microarrays, e.g., by masking (Maskos and Southern, Nuc. Acids Res. 1992,20:1679-1684), may also be used. In principal, any type of array, for example, dot blots on a nylon hybridization membrane (see Sambrook et al., Molecular Cloning A Laboratory Manual (2nd Ed.), Vol. 1-3, Cold Spring Harbor Laboratory, Cold Spring Harbor, N.Y., 1989) could be used, although, as will be recognized by those of skill in the art. For these assays nucleic acid hybridization and wash conditions are chosen so that the attached oligonucleotides specifically hybridize to at least a portion of the FGFR2 gene present in the tested sample sequence but does not hybridize to a site with a non-complementary nucleic acid sequence. The terms “hybridize” and “bind” are used interchangeably.

Alternatively, one may use allele specific hybridization to detect the mutant. In allele-specific hybridization, oligonucleotide sequences representing all possible variations at a polymorphic site are included on a DNA chip. The chip and sample are subject to conditions under which the labeled sample DNA will only bind to an oligonucleotide with an exact sequence match. In allele-specific primer extension, sample DNA hybridized to the chip may be used as a synthesis template with the affixed oligonucleotide as a primer. Under this method, only the added dNTP's are labeled. Incorporation of the labeled dNTP then serves as the signal indicating the presence of the allele. The fluorescent label may be detected by any of a number of instruments configured to read at least four different fluorescent labels on a DNA chip. In another alternative, the identity of the final dNTP added to the oligonucleotide may be assessed by mass spectrometry. In this alternative, the dNTP's may, but need not be labeled with a label of known molecular weight.

One polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al, supra, and Chee et al., Science 1996,274:610-614).

A variety of methods are available for detection and analysis of the hybridization events. Depending on the label used, detection and analysis may be carried out, for example fluorimetrically, colorimetrically or by autoradiography. By observing and measuring emitted radiation, such as fluorescent radiation or a particle emission, information may be obtained about the hybridization events. When fluorescently labeled probes are used, the fluorescence emissions at each site of transcript array can be detected by, for example, scanning confocal laser microscopy. In scanning confocal laser microscopy, a separate scan using the appropriate excitation line, is carried out for each of at least two fluorophores used to label probes. Alternatively, a laser that allows simultaneous specimen illumination at wavelengths specific to the two fluorophores and emissions from the two fluorophores may be used (see Shalon et al., Genome Res. 1996, 6:639-695).

One may also detect mutations in the FGFR2 protein, or assess dysregulated expression of the FGFR2 protein. FGFR2 may be detected by immunoassay. For example, Western blotting permits detection of a specific variant, or the presence or absence of FGFR2 expression. In particular, an immunoassay is capable of detecting a specific amino acid sequence in a FGFR2 protein. Other examples of immunoassays include ELISA. In ELISA assays, an antibody raised against whole FGFR2, or a fragment of FGFR2, or any mutant form of FGFR2 is immobilized onto a solid surface capable of binding proteins nonspecifically. One example of such a surface is polystyrene. Alternatively, purified FGFR2 or FGFR2 mutant, or any fragment thereof is immobilized onto the solid surface directly. After washing to remove incompletely adsorbed polypeptides, a blocking protein such as a solution of bovine serum albumin (BSA) or whole serum may be added to the selected surface. This allows for blocking of nonspecific adsorption sites on the immobilizing surface and thus reduces the background caused by nonspecific bindings of antibodies onto the surface. The surface with the immobilized antibodies is then contacted with a sample and incubated under conditions that facilitate immune complex (antigen/antibody) formation. Examples of such conditions include dilution of the sample with one or more diluents solutions of BSA, bovine gamma globulin (BGG) and/or phosphate buffered saline—detergent such as PBS/Tween and incubating the sample from 30 minutes to 72 hours at temperatures from 4 to 37 degrees C.

Following incubation, the surface is washed to remove nonimmunocomplexed material. The washing procedure may include washing with a solution, such as PBS/Tween or borate buffer. Following formation of specific immunocomplexes between the test sample and the bound antibody, and subsequent washing, the occurrence, and an even amount of immunocomplex formation may be determined by subjecting the immunocomplex to a second antibody against FGFR2 mutants, that recognizes a mutated epitope on the protein. In general, the second antibody may have an associated activity such as an enzymatic activity that will generate, for example, a color development upon incubating with an appropriate chromogenic substrate. Alternatively, the second antibody may be labeled with a small molecule such as biotin and the enzymatic activity linked to a ligand for the small molecule, such as streptavidin.

Quantification of FGFR2 in the sample may then be achieved by measuring the degree of color generation using, for example, a visible spectra spectrophotometer. Examples of the enzyme to which the second antibody is conjugated include but are not limited to peroxidase and alkaline phosphatase. Examples of the substrate include a peroxidase substrate such as tetramethylbenzidine or any other substrate that changes the color or another property of a solution in response to the presence of a particular enzyme. The test protein concentration may be determined by comparison with a standard curve. These protocols are detailed in Current Protocols in Molecular Biology, V. 2 Ch. 11 and Antibodies, a Laboratory Manual, Ed Harlow, David Lane, Cold Spring Harbor Laboratory (1988) pp 579-593.

Other examples of immunoassays that may be used to detect mutant forms of FGFR2 protein include radioimmunoassay, sandwich immunoassays, immunoradiometric assays, gel diffusion precipitin reactions, immunodiffusion asays, in situ immuoassays or immunohistochemistry assays (IHC), precipitation reactions, agglutination assays, complement fixation assays, immunofluorescence assays, protein A assays, immunoelectrophoresis assays, flow cytometry based assays or any other technique now known or yet to be developed that utilizes a specific antibody to detect mutant FGFR2.

Antibodies to be used in immunoassays that detect the presence of mutant forms of FGFR2 may be produced by any of a number of techniques that include but are not limited to the techniques below. Such antibodies include but are not limited to polyclonal, monoclonal, chimeric, single chain, Fab fragments, Fab expression library, and for example, humanized antibodies.

Various procedures known in the art may be used for the production of polyclonal or monoclonal antibodies to FGFR2 polypeptides or derivative or analog thereof. For the production of antibody, various host animals can be immunized by injection with the antigenic polypeptide, including but not limited to rabbits, mice, rats, sheep, goats, chickens, etc. For preparation of monoclonal antibodies directed toward the FGFR2 polypeptides, any technique that provides for the production of antibody molecules by continuous cell lines in culture may be used.

These include but are not limited to the hybridoma technique originally developed by Kohler and Milstein (Nature 256:495497, 1975), as well as the trioma technique, the human B-cell hybridoma technique (Kozbor et aI., Immunology Today 4:72, 1983; Cote et at, Proc. NatI. Acad. Sci. U.S.A. 80:2026-2030, 1983), and the EBV-hybridoma technique to produce human monoclonal antibodies (Cole et aI., in Monoclonal Antibodies and Cancer Therapy, Alan R. Liss, Inc., pp. 77-96, 1985). In an additional embodiment of the invention, monoclonal antibodies can be produced in germ-free animals (International Patent Publication No. WO 89/12690, published Dec. 28, 1989).

Techniques described for the production of single chain antibodies (U.S. Pat. Nos. 5,476,786 and 5,132,405 to Huston; U.S. Pat. No. 4,946,778) may be adapted to produce FGFR2 polypeptide-specific single chain antibodies. Alternatively the techniques described for the construction of Fab expression libraries (Huse et al., Science 246:1275-1281, 1989) may be used to allow rapid and easy identification of monoclonal Fab fragments with specificity for a FGFR2 polypeptide, or its derivatives, or analogs.

Antibody fragments which contain the idiotype of the antibody molecule may be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc.

Any biochemical assay can be used to detect expression, or accumulation of FGFR2 protein, e.g., by detecting the presence or absence of a band in samples analyzed by polyacrylamide gel electrophoresis; by the presence or absence of a chromatographic peak in samples analyzed by any of the various methods of high performance liquid chromatography, including reverse phase, ion exchange, and gel permeation; by the presence or absence of FGFR2 in analytical capillary electrophoresis chromatography, or any other quantitative or qualitative biochemical technique known in the art.

The presence or absence of mutant FGFR2 may be used to predict the presence or absence of a particular physiological characteristic. Prediction of a cellular or physiological characteristic includes the prediction of any cellular or physiological state that may be predicted by assessing the expression of a marker. Examples include the identity of a cell as a particular cell including a particular normal or cancer cell type, the likelihood that one or more diseases is present or absent, the likelihood that a present disease will progress, remain unchanged, or regress, the likelihood that a disease will respond or not respond to a particular therapy, or any other disease outcome. Further examples include the likelihood that a cell will move, senesce, apoptose, differentiate, metastasize, or change from any state to any other state or maintain its current state.

One type of cellular or physiological characteristic is the risk that a particular disease outcome will occur. Assessing this risk includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

Determining whether or not the presence or absence of an FGFR2 mutation signifies a physiological or cellular characteristic may be assessed by any of a number of methods. In assessing disease outcome or the effect of treatment, a population of patients, all of which have, a disease such as cancer, may be followed for a period of time. After the period of time expires, the population may be divided into two or more groups. For example, the population may be divided into a first group of patients whose disease progresses to a particular endpoint and a second group of patients whose disease does not progress to the particular endpoint. Examples of endpoints include disease recurrence, death, metastasis or other states to which disease may progress. If presence or absence of an FGFR2 mutation in a sample is more similar to the predetermined expression of the marker in one group relative to the other group, the sample may be assigned a risk of having the same outcome as the patient group to which it is more similar.

For example, Receiver Operating Characteristic curves, or “ROC” curves, may be calculated by plotting the value of a variable versus its relative frequency in two populations. For any particular marker, a distribution of marker expression levels for subjects with and without a disease may overlap. This indicates that the test does not absolutely distinguish between the two populations with complete accuracy. The area of overlap indicates where the test cannot distinguish the two groups. A threshold is selected. Expression of the marker in the sample above the threshold indicates the sample is similar to one group and expression of the marker below the threshold indicates the sample is similar to the other group. The area under the ROC curve is a measure of the probability that the expression correctly indicated the similarity of the sample to the proper group. See, e.g., Hanley et al., Radiology 143: 29-36 (1982) hereby incorporated by reference.

Other methods may be used to assess how accurately the presence or absence of an FGFR2 mutation signifies a particular physiological or cellular characteristic. Such methods include a positive likelihood ratio, negative likelihood ratio, odds ratio, and/or hazard ratio. In the case of a likelihood ratio, the likelihood that the expression of the marker would be found in a sample with a particular cellular or physiological characteristic is compared with the likelihood that the expression of the marker would be found in a sample lacking the particular cellular or physiological characteristic.

An odds ratio measures effect size and describes the amount of association or non-independence between two groups. An odds ratio is the ratio of the odds of a marker being expressed in one set of samples versus the odds of the marker being expressed in the other set of samples. An odds ratio of 1 indicates that the event or condition is equally likely to occur in both groups. An odds ratio grater or less than 1 indicates that expression of the marker is more likely to occur in one group or the other depending on how the odds ratio calculation was set up. A hazard ratio may be calculated by estimate of relative risk. Relative risk is the chance that a particular event will take place. It is a ratio of the probability that an event such as development or progression of a disease will occur in samples that exceed a threshold level of expression of a marker over the probability that the event will occur in samples that do not exceed a threshold level of expression of a marker. Alternatively, a hazard ratio may be calculated by the limit of the number of events per unit time divided by the number at risk as the time interval decreases. In the case of a hazard ratio, a value of 1 indicates that the relative risk is equal in both the first and second groups; a value greater or less than 1 indicates that the risk is greater in one group or another, depending on the inputs into the calculation.

Additionally, multiple threshold levels of expression may be determined. This can be the case in so-called “tertile,” “quartile,” or “quintile” analyses. In these methods, multiple groups can be considered together as a single population, and are divided into 3 or more bins having equal numbers of individuals. The boundary between two of these “bins” may be considered threshold levels of expression indicating a particular level of risk of a disease developing or signifying a physiological or cellular state. A risk may be assigned based on which “bin” a test subject falls into.

The present invention further provides kits for the determination of the sequence within the FGFR2 gene in a subject to diagnose or classify endometrial cancer. Kits include any combination of components that facilitates the performance of an assay. A kit that facilitates detection of mutant FGFR2 may include suitable nucleic acid-based and immunological reagents as well as suitable buffers, control reagents, and printed protocols.

Kits that facilitate nucleic acid based methods may further include one or more of the following: specific nucleic acid probes or primers such as sequencing primers, labeling reagents, and reagents that facilitate hybridization.

Kits that facilitate antibody based methods of detecting mutant FGFR2 proteins may further include one or more of the following: a labeled or unlabeled antibody with specificity to an FGFR2 mutant, a labeled secondary antibody, and an enzyme substrate.

A kit may also contain an indication of a result that signifies a particular physiological or cellular characteristic. An indication includes any result that, using the kit in which the indication is provided, would signal the presence or absence of any physiological or cellular state that the kit is configured to detect. The indication may be expressed numerically, as a nucleic acid or protein sequence, expressed as a color, expressed as an intensity of a band, derived from a standard curve, or compared to a control. The indication may be printed on a writing that may be included in the kit or it may be posted on the internet or embedded in a software package.

### EXAMPLE

476 frozen endometrioid endometrial tumors collected at the Washington University University School of Medicine were examined for mutation in FGFR2 by direct sequencing. The relationship between FGFR2 mutations status and clinicopathological variables including overall and progression free survival were evaluated using Kaplan-Meier survival analysis and Cox proportional hazard models.

FGFR2 mutations were detected in 49/476 (10%) of cases. FGFR2 mutations were more common in FIGO grade 1 and 2 tumors than grade 3 tumors (p<0.03) and were associated with microsatellite instability (P=0.01). Mutation of FGFR2 was not significantly associated with age at diagnosis, tumor stage, or overall or progression free survival. However, in women with early stage, intermediate risk disease (314 cases) univariate analysis found that FGFR2 mutation was associated with decreased progression free survival (hazard ratio [HR]=2.51; 95% CI, 1.10 to 5.77; p=0.03) and decreased overall survival (HR 2.00; 95% CI 1.08 to 3.68; p=0.03;). Furthermore, multivariate analysis revealed that FGFR2 mutation had independent prognostic value (HR 3.04; 95% CI, 1.26 to 7.35; p=0.03) in the cohort of women with an intermediate risk of recurrence.

FGFR2 mutation is associated with worse prognosis in patients with early stage, intermediate risk endometrial tumors.

Since 1991, the Division of Gynecologic Oncology at Washington University School of Medicine (St Louis, Mo.) has prospectively collected tumor samples from patients undergoing a hysterectomy for suspected uterine cancer. For all cases, surgery was performed by a gynecologic oncologist at Washington University School of Medicine/Barnes—Jewish Hospital. Surgical staging and tumor grade was assigned on the basis of International Federation of Gynecology and Obstetrics (FIGO) 1988 criteria by experienced gynecologic pathologists. None of these patients underwent preoperative radiation or chemotherapy. All participants consented to molecular analyses and follow-up monitoring. All prospectively collected clinical and pathologic information was stored in a computerized database. Following their initial treatment, these patients were typically followed at 3-month intervals for the first 2 years, then at 6-month intervals for at least 2 years, and then annually thereafter. Disease surveillance included physical examination and periodic vaginal cuff cytology. Diagnostic imaging and directed biopsies were performed as clinically indicated. Histological confirmation of all recurrences was performed when appropriate. Follow-up data were extracted from clinic charts, hospital records, and the Barnes-Jewish Hospital/Siteman Cancer Center's tumor registry surveillance database.

Within this cohort there were 476 patients with endometrioid endometrial cancer that were informative for survival analyses. Of this group, there were 314 cases classified as early stage, intermediate risk. For the purposes of this study, intermediate risk was set at Stage 1 (G3), Stage IB, IC, IIA, IIB (G1-G3).

Tissue specimens and blood were obtained at the time of surgery, snap frozen, and stored at −70° C. Tumors were evaluated to select tissues with >66% neoplastic cellularity for DNA preparations. DNA was isolated using proteinase K and phenol extraction or through the use of a commercially available kit. DNA was extracted from peripheral blood leukocytes as previously described. When blood was not available, normal DNA was extracted from uninvolved myometrium (See References 10 and 11)

Exons 7, 8, 10, 13 and 15 of FGFR2 (See FIG. 1) were tested for mutations by direct DNA sequencing. The M13 tailed PCR primers and conditions used were essentially as previously described (See Reference 8). Sequences were analyzed using Sequencher (Gene Codes). All potential mutations were confirmed with repeat amplification and sequencing of the exon of interest. Matched normal DNA was analyzed to confirm the mutation arose somatically.

The relationship between FGFR2 mutation status and covariates was performed using Fisher's exact test or Student's t-test as appropriate. Overall survival (OS) was defined as the time from date of surgery to death due to any cause. Survivors were censored at the date of last contact. Disease free survival (DFS) was defined as the time from surgery to recurrence or progression. The Kaplan-Meier product limit method was used to estimate OS and DFS. Univariate and multivariate Cox proportional hazard models were fitted to assess the effects of the covariates on OS and DFS, and the proportional hazard assumptions were checked using scaled Schoenfeld residuals (See Reference 12). In the analysis of DFS, Gray's competing risk methods were also used to account for the potential competing effect of death (See Reference 13). All analyses were two-sided and significance was set at a P-value of 0.05. Statistical analyses were performed using SAS (SAS Institutes, Cary, N.C.), as well as the cmprsk R (http://biowww.dfci.harvard.edu/˜gray) statistical packages for competing risk analysis.

A total of 476 surgically staged endometrioid endometrial cancers that were informative for survival analyses were assessed for FGFR2 mutations (Table 1). The mean age at diagnosis was 63.6 years with a mean follow-up time of 68 months (0.7-176). The majority of patients presented with early-stage disease (394 or 83% stage I or II). Among those, 314 were considered to have an intermediate risk of recurrence based on stage and grade (IA G3; IB, IC, IIA, IIB, G1-G3). The mean age at diagnosis in this group was 64.7 year, all patients were >2 years post surgery and the mean time of follow-up was 72 months (0.7-176).

Overall, we have identified mutations in 49/476 (10.3%) endometrial tumors with endometrioid histology (Table 2), including those originally reported in 116 of these cases (See Reference 8). One FGFR2 sequence alteration (frameshift) originally reported as a mutation was excluded because of uncertainty as to whether the sequence change is pathogenic. The most common mutations were S252W (n=18; 37%) and N550K (n=12, 25%). All together, 7 mutations affecting 6 codons (S252W, P253R, Y376C, C383R, N550K, N550H and K660E) account for 90% of the mutations identified. We identified two additional novel mutations in the transmembrane domain not previously described (V396D and L398M).

There was no association between FGFR2 mutation and stage (stage I, 9%, stage II, 18%, stage III/IV, 10%) or age at diagnosis. FGFR2 mutations were more common in Caucasian/Asian cases (45/420, 11%) than African American patients (2/56, 3%) but the difference was not statistically significant (p=0.10). FGFR2 mutation was, however, significantly associated with grade. Mutations were more common in well (FIGO grade 1) and moderately differentiated (grade 2) tumors (29/250 and 18/156; 11.5%) compared to poorly differentiated (grade 3) tumors (2/69; 3%) (p<0.03). FGFR2 mutation was strongly associated with defective DNA mismatch repair (tumor MSI). Twenty-five of 159 MSI-positive cases had FGFR2 mutations (15.7%) whereas 24 of 316 MSI-stable cases (7.6%) had mutations (P=0.01).

In the entire cohort, univariate analyses revealed shorter progression free survival (PFS) and overall survival (OS) is associated with advanced stage (III/IV) (p<0.0001) and a poorly differentiated tumors-FIGO grade 3 (p<0.0001). FGFR2 mutations are not significantly associated with overall or progression free survival (p<0.29). Multivariate analysis revealed age, stage and grade were significantly associated with poor PFS and OS (Table 3).

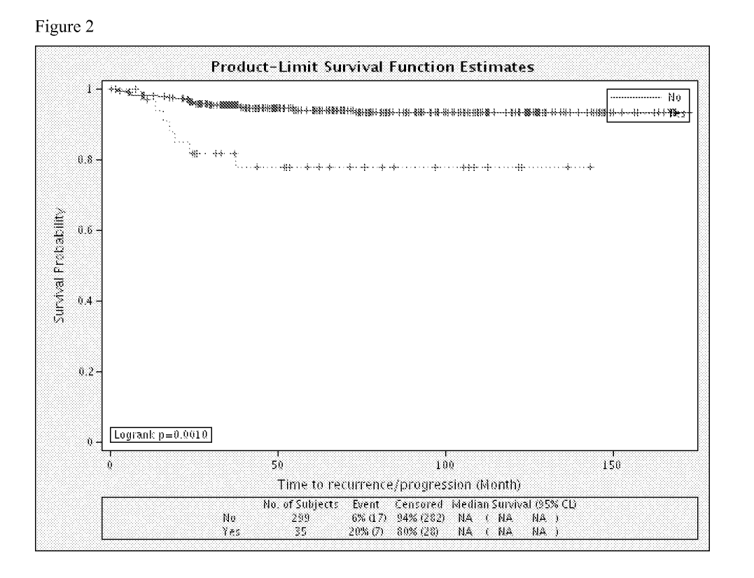

FGFR2 mutation is associated with outcome in patients with so-called intermediate risk tumors, the 314 stage 1A (G3), IB, IC or II cases that comprise 66% of our cohort. FGFR2 mutations were detected in 33/314 (10.5%) of these intermediate risk cases. FGFR2 mutations were more common in those patients that recurred (7/35; 20%) versus those that did not (26/279; 9.3%). Univariate analysis revealed FGFR2 was significantly associated with decreased progression free survival (HR=2.51; 95% CI 1.10-5.77; P=0.03) and decreased overall survival (HR=2.00; 95% CI 1.08-3.68; P=0.03). Kaplan Meier survival plots for PFS and OS according to FGFR2 mutation status are presented in FIG. 2. Consistent with the literature, a poorly differentiated histology was associated with reduced PFS (p<0.0042) and OS (P<0.0002) (See References 1, 18). Several other clinicopathological variables showed a weak association with PFS and OS respectively including: age (p<0.06; p<0.06), race (p<0.26; p<0.11) and stage II (p<0.16; p<0.06).

Multivariate analysis revealed that FGFR2 demonstrated independent prognostic value to that provided by the existing clinicopathologic features of age, stage, grade and race (HR=3.04, C.I. 1.26-7.35) in the cohort of 314 patients with an intermediate risk of recurrence.

### Tables

TABLE 1
Sample description

| | Entire cohort | | Cohort of 314 | |
|---|---|---|---|---|
| | of 484 | | intermediate risk | |
| | Endometrioid | | Endometrioid | |
| | Endometrial | | Endometrial | |
| | Cancers (WashU) | | Cancers (WashU) | |
| Mean Age at Dx (SD) | 63.4 | (11.7) | 64.7 | (11.2) |
| Follow-up time (mean) | 68 months | (0.7-176) | 72 months | (0.7-176) |

Race

| | | | | |
|---|---|---|---|---|
| Caucasian/Asian | 420 | (88%) | 277 | (88%) |
| African American | 56 | (12%) | 37 | (12%) |

FIGO stage

| | | | | |
|---|---|---|---|---|
| IA | 82 | (17%) | 2 | (1%) |
| IB | 202 | (42%) | 202 | (64%) |
| 1C | 71 | (15%) | 71 | (23%) |
| IIA | 16 | (3%) | 16 | (5%) |
| IIB | 23 | (5%) | 23 | (7%) |

| | | | |
|---|---|---|---|
| III | 66 | (13%) | — |
| IV | 16 | (2%) | — |

Grade

| | | | | |
|---|---|---|---|---|
| 1 | 250 | (53%) | 163 | (52%) |
| 2 | 157 | (33%) | 108 | (34%) |
| 3 | 69 | (14%) | 43 | (14%) |

Recurrence

| | | | | |
|---|---|---|---|---|
| No | 406 | (85%) | 279 | (89%) |
| Yes | 70 | (15%) | 35 | (11%) |

Vital Status

| | | | | |
|---|---|---|---|---|
| Alive | 327 | (69%) | 226 | (72%) |
| Dead | 149 | (31%) | 88 | (28%) |

MSI

| | | | | |
|---|---|---|---|---|
| No | 317 | (67%) | 203 | (65%) |
| Yes | 159 | (33%) | 111 | (35%) |

TABLE 2
Clinicopathological features of endometrial
tumors with FGFR2 mutations

| Case ID | Stage | Grad | Recur | FGFR2b Nucleotide^{a} | FGFR2 |
|---|---|---|---|---|---|
| 1133 | IA | 1 | N | c.755C > G | p.S252W |
| 1141 | IB | 2 | N | c.755C > G | p.S252W |
| 1195 | IA | 1 | N | c.755C > G | p.S252W |
| 1410 | IIIC | 2 | N | c.755C > G | p.S252W |
| 1431 | IIA | 1 | N | c.755C > G | p.S252W |
| 1536 | IA | 1 | N | c.755C > G | p.S252W |
| 1604 | IA | 1 | N | c.755C > G | p.S252W |
| 1806 | IB | 1 | N | c.755C > G | p.S252W |
| 1829 | IC | 1 | N | c.755C > G | p.S252W |
| 1958 | IC | 1 | Y | c.755C > G | p.S252W |
| 1987 | IB | 1 | N | c.755C > G | p.S252W |
| 1359^{c} | IB | 2 | Y | c.755C > G | p.S252W |
| 1574^{c} | IC | 2 | Y | c.755C > G | p.S252W |
| 1484^{c} | IIIC | 3 | Y | c.755C > G | p.S252W |
| 1316^{c} | IIIC | 1 | Y | c.755C > G | p.S252W |
| 1792^{c} | IIIC | 1 | N | c.755C > G | p.S252W |
| 1482^{c} | IVA | 2 | N | c.755C > G | p.S252W |
| 1130 | IC | 1 | N | c.758C > G | p.P253R |
| 1590 | IB | 2 | N | c.758C > G | p.P253R |
| 1684^{c} | IB | 1 | N | c.1118C > G | p.S373C |
| 1363 | IB | 2 | N | c.1127A > G | p.Y376C |
| 1655^{c} | IIIC | 2 | Y | c.1127A > G | p.Y376C |
| 1361^{c} | IB | 1 | Y | c.1175T > G | p.M392R |
| 2033 | IB | 1 | N | c.1187 1188delinsAT | p.V396D |
| 1524 | IC | 2 | N | c.1192C > A | p.L398M |
| 1744^{c} | IIIC | 2 | N | c.1642A > G | p.I548V |
| 1220 | IB | 1 | N | c.1650T > A | p.N550K |
| 1231 | IB | 1 | N | c.1650T > A | p.N550K |
| 1249 | IB | 1 | N | c.1650T > A | p.N550K |
| 1347 | IB | 1 | N | c.1650T > A | p.N550K |
| 1464 | IIA | 3 | N | c.1650T > A | p.N550K |
| 1631 | IIB | 1 | N | c.1650T > A | p.N550K |
| 1714 | IA | 1 | N | c.1650T > A | p.N550K |
| 1877 | IIA | 1 | N | c.1650T > A | p.N550K |
| 1946 | IIB | 1 | N | c.1650T > G | p.N550K |
| 1267^{c} | IIA | 2 | Y | c.1650T > A | p.N550K |
| 1391^{c} | IIIC | 2 | N | c.1650T > A | p.N550K |
| 1528^{c} | IVA | 2 | N | c.1650T > A | p.N550K |
| 2056 | IB | 2 | N | c.1648A > C | p.N550H |
| 2066 | IB | 2 | N | c.1648A > C | p.N550H |
| 1550 | IB | 1 | N | c.1978A > G | p.K660E |
| 1587 | IC | 1 | N | c.1978A > G | p.K660E |
| 2024 | IB | 2 | N | c.1978A > G | p.K660E |
| 1717^{c} | IC | 2 | N | c.1978A > G | p.K660E |
| 1164 | IC | 2 | N | c.1147T > C | p.C383R |
| 1729 | IA | 1 | N | c.1147T > C | p.C383R |
| 1094^{c} | IB | 1 | Y | c.1147T > C | p.C383R |
| 1492^{c} | IC | 1 | Y | c.┌755C > | p.┌S252W(+) |
| | | | | GC755G(+)1127A > G┐ | Y376C┐ |
| 1272^{c} | IA | 1 | N | Intron10 A > C + 2 | |

^{a}Numbering relative to NM_022970.2
^{b}Numbering relative to NP_075259.2
^{c}These mutations have been reported previously (See Reference 8).

TABLE 3
Multivariate Analysis

| | Entire Intermediate Risk Cohort (n = 315) | | | | | |
|---|---|---|---|---|---|---|
| | Progression Free Survival | | | Overall Survival | | |
| | HR | 95% Cl | P | HR | 95% Cl | P |
| Age | 1.02 | 0.99-1.05 | 0.167 | 1.04 | 1.02-1.06 | 0.0002* |
| FIGO stage II | 1.66 | 0.71-3.86 | 0.244 | 1.91 | 1.09-3.34 | 0.025* |
| FIGO grade 2 | 1.68 | 0.76-3.69 | 0.200 | 1.71 | 1.05-2.78 | 0.031* |
| FIGO grade 3 | 3.53 | 1.46-8.54 | 0.005* | 2.68 | 1.47-4.89 | 0.001* |
| Race (Black) | 1.61 | 0.66-3.94 | 0.300 | 1.38 | 0.76-2.50 | 0.291 |
| FGFR2 | 3.04 | 1.26-7.35 | 0.014* | 2.27 | 1.20-4.31 | 0.012* |

TABLE 4
FIGO Staining Classifications and Risk of Recurrence

| Stage | Grade 1 | Grade 2 | Grade 3 |
|---|---|---|---|
| IA—Tumor limited to endometrium | Low risk | Low risk | High-int risk |
| IB—Invasion to less than ½ myometrium | Low-int risk | Low-int risk | High-int risk |
| IC—Invasion to more than ½ myometrium | Low-int risk | Low-int risk | High-int risk |
| IIA—Endocervical glandular involvement | Low-int risk | Low-int risk | High-int risk |
| only | | | |
| IIB—Cervical stromal invasion | High-int risk | High-int risk | High-int risk |
| IIIA—Tumor invades serosa and/or adnexa | High risk | High risk | High risk |
| and/or positive peritoneal cytology | | | |
| IIIB—Metastases to pelvic and/or paraaortic | High risk | High risk | High risk |
| lymph nodes | | | |
| IVA—Tumor invasion of bladder and/or | High risk | High risk | High risk |
| bowel mucosa | | | |
| IVB—Distant metastases including | High risk | High risk | High risk |
| intraabdominal and/or inguinal lymph nodes | | | |

### SEQUENCES

| SEQ ID NO 1: | |
|---|---|
| ggcggcggct ggaggagagc gcggtggaga gccgagcggg cgggcggcgg gtgcggagcg | 60 |
| ggcgagggag cgcgcgcggc cgccacaaag ctcgggcgcc gcggggctgc atgcggcgta | 120 |
| cctggcccgg cgcggcgact gctctccggg ctggcggggg ccggccgcga gccccggggg | 180 |
| ccccgaggcc gcagcttgcc tgcgcgctct gagccttcgc aactcgcgag caaagtttgg | 240 |
| tggaggcaac gccaagcctg agtcctttct tcctctcgtt ccccaaatcc gagggcagcc | 300 |
| cgcgggcgtc atgcccgcgc tcctccgcag cctggggtac gcgtgaagcc cgggaggctt | 360 |
| ggcgccggcg aagacccaag gaccactctt ctgcgtttgg agttgctccc cgcaaccccg | 420 |
| ggctcgtcgc tttctccatc ccgacccacg cggggcgcgg ggacaacaca ggtcgcggag | 480 |
| gagcgttgcc attcaagtga ctgcagcagc agcggcagcg cctcggttcc tgagcccacc | 540 |
| gcaggctgaa ggcattgcgc gtagtccatg cccgtagagg aagtgtgcag atgggattaa | 600 |
| cgtccacatg gagatatgga agaggaccgg ggattggtac cgtaaccatg gtcagctggg | 660 |
| gtcgtttcat ctgcctggtc gtggtcacca tggcaacctt gtccctggcc cggccctcct | 720 |
| tcagtttagt tgaggatacc acattagagc cagaagagcc accaaccaaa taccaaatct | 780 |
| ctcaaccaga agtgtacgtg gctgcgccag gggagtcgct agaggtgcgc tgcctgttga | 840 |
| aagatgccgc cgtgatcagt tggactaagg atggggtgca cttggggccc aacaatagga | 900 |
| cagtgcttat tggggagtac ttgcagataa agggcgccac gcctagagac tccggcctct | 960 |
| atgcttgtac tgccagtagg actgtagaca gtgaaacttg gtacttcatg gtgaatgtca | 1020 |
| cagatgccat ctcatccgga gatgatgagg atgacaccga tggtgcggaa gattttgtca | 1080 |
| gtgagaacag taacaacaag agagcaccat actggaccaa cacagaaaag atggaaaagc | 1140 |
| ggctccatgc tgtgcctgcg gccaacactg tcaagtttcg ctgcccagcc ggggggaacc | 1200 |
| caatgccaac catgcggtgg ctgaaaaacg ggaaggagtt taagcaggag catcgcattg | 1260 |
| gaggctacaa ggtacgaaac cagcactgga gcctcattat ggaaagtgtg gtcccatctg | 1320 |
| acaagggaaa ttatacctgt gtagtggaga atgaatacgg gtccatcaat cacacgtacc | 1380 |
| acctggatgt tgtggagcga tcgcctcacc ggcccatcct ccaagccgga ctgccggcaa | 1440 |
| atgcctccac agtggtcgga ggagacgtag agtttgtctg caaggtttac agtgatgccc | 1500 |
| agccccacat ccagtggatc aagcacgtgg aaaagaacgg cagtaaatac gggcccgacg | 1560 |
| ggctgcccta cctcaaggtt ctcaaggccg ccggtgttaa caccacggac aaagagattg | 1620 |
| aggttctcta tattcggaat gtaacttttg aggacgctgg ggaatatacg tgcttggcgg | 1680 |
| gtaattctat tgggatatcc tttcactctg catggttgac agttctgcca gcgcctggaa | 1740 |
| gagaaaagga gattacagct tccccagact acctggagat agccatttac tgcatagggg | 1800 |
| tcttcttaat cgcctgtatg gtggtaacag tcatcctgtg ccgaatgaag aacacgacca | 1860 |
| agaagccaga cttcagcagc cagccggctg tgcacaagct gaccaaacgt atccccctgc | 1920 |
| ggagacaggt aacagtttcg gctgagtcca gctcctccat gaactccaac accccgctgg | 1980 |
| tgaggataac aacacgcctc tcttcaacgg cagacacccc catgctggca ggggtctccg | 2040 |
| agtatgaact tccagaggac ccaaaatggg agtttccaag agataagctg acactgggca | 2100 |
| agcccctggg agaaggttgc tttgggcaag tggtcatggc ggaagcagtg ggaattgaca | 2160 |
| aagacaagcc caaggaggcg gtcaccgtgg ccgtgaagat gttgaaagat gatgccacag | 2220 |
| agaaagacct ttctgatctg gtgtcagaga tggagatgat gaagatgatt gggaaacaca | 2280 |
| agaatatcat aaatcttctt ggagcctgca cacaggatgg gcctctctat gtcatagttg | 2340 |
| agtatgcctc taaaggcaac ctccgagaat acctccgagc ccggaggcca cccgggatgg | 2400 |
| agtactccta tgacattaac cgtgttcctg aggagcagat gaccttcaag gacttggtgt | 2460 |
| catgcaccta ccagctggcc agaggcatgg agtacttggc ttcccaaaaa tgtattcatc | 2520 |
| gagatttagc agccagaaat gttttggtaa cagaaaacaa tgtgatgaaa atagcagact | 2580 |
| ttggactcgc cagagatatc aacaatatag actattacaa aaagaccacc aatgggcggc | 2640 |
| ttccagtcaa gtggatggct ccagaagccc tgtttgatag agtatacact catcagagtg | 2700 |
| atgtctggtc cttcggggtg ttaatgtggg agatcttcac tttagggggc tcgccctacc | 2760 |
| cagggattcc cgtggaggaa ctttttaagc tgctgaagga aggacacaga atggataagc | 2820 |
| cagccaactg caccaacgaa ctgtacatga tgatgaggga ctgttggcat gcagtgccct | 2880 |
| cccagagacc aacgttcaag cagttggtag aagacttgga tcgaattctc actctcacaa | 2940 |
| ccaatgagga atacttggac ctcagccaac ctctcgaaca gtattcacct agttaccctg | 3000 |
| acacaagaag ttcttgttct tcaggagatg attctgtttt ttctccagac cccatgcctt | 3060 |
| acgaaccatg ccttcctcag tatccacaca taaacggcag tgttaaaaca tgaatgactg | 3120 |
| tgtctgcctg tccccaaaca ggacagcact gggaacctag ctacactgag cagggagacc | 3180 |
| atgcctccca gagcttgttg tctccacttg tatatatgga tcagaggagt aaataattgg | 3240 |
| aaaagtaatc agcatatgtg taaagattta tacagttgaa aacttgtaat cttccccagg | 3300 |
| aggagaagaa ggtttctgga gcagtggact gccacaagcc accatgtaac ccctctcacc | 3360 |
| tgccgtgcgt actggctgtg gaccagtagg actcaaggtg gacgtgcgtt ctgccttcct | 3420 |
| tgttaatttt gtaataattg gagaagattt atgtcagcac acacttacag agcacaaatg | 3480 |
| cagtatatag gtgctggatg tatgtaaata tattcaaatt atgtataaat atatattata | 3540 |
| tatttacaag gagttatttt ttgtattgat tttaaatgga tgtcccaatg cacctagaaa | 3600 |
| attggtctct ctttttttaa tagctatttg ctaaatgctg ttcttacaca taatttctta | 3660 |
| attttcaccg agcagaggtg gaaaaatact tttgctttca gggaaaatgg tataacgtta | 3720 |
| atttattaat aaattggtaa tatacaaaac aattaatcat ttatagtttt ttttgtaatt | 3780 |
| taagtggcat ttctatgcag gcagcacagc agactagtta atctattgct tggacttaac | 3840 |
| tagttatcag atcctttgaa aagagaatat ttacaatata tgactaattt ggggaaaatg | 3900 |
| aagttttgat ttatttgtgt ttaaatgctg ctgtcagacg attgttctta gacctcctaa | 3960 |
| atgccccata ttaaaagaac tcattcatag gaaggtgttt cattttggtg tgcaaccctg | 4020 |
| tcattacgtc aacgcaacgt ctaactggac ttcccaagat aaatggtacc agcgtcctct | 4080 |
| taaaagatgc cttaatccat tccttgagga cagaccttag ttgaaatgat agcagaatgt | 4140 |
| gcttctctct ggcagctggc cttctgcttc tgagttgcac attaatcaga ttagcctgta | 4200 |
| ttctcttcag tgaattttga taatggcttc cagactcttt ggcgttggag acgcctgtta | 4260 |
| ggatcttcaa gtcccatcat agaaaattga aacacagagt tgttctgctg atagttttgg | 4320 |
| ggatacgtcc atctttttaa gggattgctt tcatctaatt ctggcaggac ctcaccaaaa | 4380 |
| gatccagcct catacctaca tcagacaaaa tatcgccgtt gttccttctg tactaaagta | 4440 |
| ttgtgttttg ctttggaaac acccactcac tttgcaatag ccgtgcaaga tgaatgcaga | 4500 |
| ttacactgat cttatgtgtt acaaaattgg agaaagtatt taataaaacc tgttaatttt | 4560 |
| tatactgaca ataaaaatgt ttctacagat attaatgtta acaagacaaa ataaatgtca | 4620 |
| cgcaacttat ttttttaata aaaaaaaaaa aaaa | 4654 |
| SEQ ID NO. 2 | |
| MVSWGRFICL VVVTMATLSL ARPSFSLVED TTLEPEEPPT KYQISQPEVY VAAPGESLEV | 60 |
| RCLLKDAAVI SWTKDGVHLG PNNRTVLIGE YLQIKGATPR DSGLYACTAS RTVDSETWYF | 120 |
| MVNVTDAISS GDDEDDTDGA EDFVSENSNN KRAPYWTNTE KMEKRLHAVP AANTVKFRCP | 180 |
| AGGNPMPTMR WLKNGKEFKQ EHRIGGYKVR NQHWSLIMES VVPSDKGNYT CVVENEYGSI | 240 |
| NHTYHLDVVE RSPHRPILQA GLPANASTVV GGDVEFVCKV YSDAQPHIQW IKHVEKNGSK | 300 |
| YGPDGLPYLK VLKAAGVNTT DKEIEVLYIR NVTFEDAGEY TCLAGNSIGI SFHSAWLTVL | 360 |
| PAPGREKEIT ASPDYLEIAI YCIGVFLIAC MVVTVILCRM KNTTKKPDFS SQPAVHKLTK | 420 |
| RIPLRRQVTV SAESSSSMNS NTPLVRITTR LSSTADTPML AGVSEYELPE DPKWEFPRDK | 480 |
| LTLGKPLGEG CFGQVVMAEA VGIDKDKPKE AVTVAVKMLK DDATEKDLSD LVSEMEMMKM | 540 |
| IGKHKNIINL LGACTQDGPL YVIVEYASKG NLREYLRARR PPGMEYSYDI NRVPEEQMTF | 600 |
| KDLVSCTYQL ARGMEYLASQ KCIHRDLAAR NVLVTENNVM KIADFGLARD INNIDYYKKT | 660 |
| TNGRLPVKWM APEALFDRVY THQSDVWSFG VLMWEIFTLG GSPYPGIPVE ELFKLLKEGH | 720 |
| RMDKPANCTN ELYMMMRDCW HAVPSQRPTF KQLVEDLDRI LTLTTNEEYL DLSQPLEQYS | 780 |
| PSYPDTRSSC SSGDDSVFSP DPMPYEPCLP QYPHINGSVK T | |

### REFERENCES

The inventors herein expressly incorporate by reference into the specification, all of the following materials to the greatest extent allowed.
1. Creutzberg C L et al, Lancet 355, 1404-1411 (2000).
2. Tangjitgamol S et al, Lancet Oncol 10, 1119-1127 (2009).
3. Lu K H, Semin Oncol 36, 137-144 (2009).
4. Cragun J M et al, J Clin Oncol 23, 3668-3675 (2005).
5. Barton D P et al, Int J Gynecol Cancer 19, 1465 (2009).
6. Benedetti Panici P et al, J Natl Cancer Inst 100, 1707-1716 (2008).
7. Kwon J S et al, Obstet Gynecol 114, 736-743 (2009).
8. Pollock P M et al, Oncogene 26, 7158-7162 (2007).
9. Dutt A et al, Proc Natl Acad Sci U S A, 105, 8713-8717 (2008).
10. Miller S A et al, Nucleic Acids Res 16, 1215 (1988).
11. Lahiri D K and Nurnberger, Jr. J I, Nucleic Acids Res, 19, 5444 (1991).
12. Grambsch P and Therneau T, Biometrika 81, 515-526 (1994).
13. Fine J P and Gray R J, J Am Stat Assoc 94, 496-509 (1999).
14. Meyers G A et al, Nat Genet 11, 462-464 (1995).
15. van Rhijn B W et al, Eur J Hum Genet 10, 819-824 (2002).
16. He L and Hristova K, J Mol Biol 384, 1130-1142 (2008).
17. Monsonego-Ornan, E et al, Mol Cell Biol 20, 516-522 (2000).
18. Keys H M et al, Gynecol Oncol 92, 744-51 (2004).
19. Tsuboi R, J Invest Dermatol 101, 49-53 (1993).
20. Putnins E E et al, Cell Adhes Commun 7, 211-221 (1999).
21. Zheng J et al, Eur J Cell Biol 69, 128-134 (1996).
22. Putnins E E et al, J Invest Dermatol 104, 989-94 (1995).
23. Putnins, E E et al, Matrix Biol 15, 21-29 (1996).
24. Madlener M et al, Biochem J 320 (Pt 2), 659-664 (1996).
25. Uitto V J et al, Am J Pathol 152, 1489-1499 (1998).
26. Nomura S et al, Br J Cancer 99, 305-313 (2008).
27. Toyokawa T et al, Oncol Rep 21, 875-880 (2009).
28. Creasman W T et al, Cancer 60, 2035-2041 (1987).
29. Morrow C P et al, Gynecol Oncol 40, 55-65 (1991).
30. Engelsen I B et al, APMIS, 117, 693-707 (2009).
31. Susini T et al, Cancer 109, 882-890 (2007).
32. Leary R J et al, Sci Transl Med 2, 20ra14 (February, 2010).
33. Pollock and Goodfellow, WO/2008/118877, international filing date Mar. 24, 2008.

We claim:

1. A method of classifying a subject into a cohort comprising:
obtaining a sample from the subject; and
subjecting the sample to conditions that allow detection of a mutant of a sequence selected from the group consisting of SEQ ID NO. 1 and SEQ ID NO. 2;
wherein the subject is known to have had endometrial cancer;
and wherein the cohort comprises two or more individuals with an increased risk of recurrence of endometrial cancer.

2. The method of claim 1 wherein the mutant comprises a mutation resulting in an amino acid change selected from the group consisting of S252W, P253R, S373C, Y376C, C383R, G385R, I548V, N550K, N550H, K660E, M392R, V396D, L398M, and IVS 10+2A>C.

3. The method of claim 2 wherein the endometrial cancer is of the endometrioid subtype.

4. The method of claim 3 wherein the stage of the sample is selected from the group consisting of Stage IA, Stage IB, Stage IC, Stage IIA, Stage IIB.

5. The method of claim 3 wherein the grade of the sample is selected from the group consisting of Grade 1 and Grade 2 and Grade 3.

6. The method of claim 1 wherein the conditions comprise detection of a mutant of SEQ ID NO. 1.

7. The method of claim 6 wherein the conditions comprise the use of a technology selected from a group consisting of: nucleic acid sequencing, microarray analysis, PCR amplification, allele specific PCR amplification, restriction fragment length polymorphism, allele specific hybridization, allele specific primer extension, and Southern blot.

8. The method of claim 1 wherein the conditions comprise detection of a mutant of SEQ ID NO. 2.

9. The method of claim 8 wherein the conditions comprise the use of a technology selected from the group consisting of HPLC, mass spectrometry, ELISA, flow cytometry, immunohistochemistry and radioimmunoassay.

10. The method of claim 8 wherein the mutant is detected by assessing the level of activity of the FGFR2 protein.

11. A kit used to classify a subject into a cohort comprising:
a first reagent capable of detecting a mutant of a sequence selected from the group consisting of SEQ ID NO. 1 and SEQ ID NO. 2; and
an indication of a result that signifies classification of the subject into the cohort wherein the cohort comprises two or more individuals with an increased risk of recurrence of endometrial cancer.

12. The kit of claim 11 wherein the mutant comprises a mutation resulting in an amino acid change selected from the group consisting of S252W, P253R, S373C, Y376C, C383R, G385R, I548V, N550K, N550H, K660E, M392R, V396D, L398M, and IVS 10+2A>C.

13. The kit of claim 12 wherein the first reagent is capable of binding a mutant of SEQ ID NO. 1.

14. The kit of claim 13 wherein the kit further comprises a component that facilitates the use of a technology selected from the group consisting of nucleic acid sequencing, microarray analysis, PCR amplification, allele specific PCR amplification, restriction fragment length polymorphism, allele specific hybridization, allele specific primer extension, and Southern blot.

15. The kit of claim 12 wherein the first reagent is capable of binding a mutant of SEQ ID NO. 2.

16. The kit of claim 15 wherein the first reagent comprises a first antibody.

17. The kit of claim 16 wherein the kit further comprises a component that facilitates the use of a technology selected from the group consisting of ELISA, flow cytometry and radioimmunoassay.

18. The kit of claim 11 wherein the result comprises a nucleic acid sequence.

19. The kit of claim 11 wherein the result comprises an optical density value.

20. The kit of claim 11 wherein the indication comprises a positive control.

21. The kit of claim 11 wherein the indication comprises a writing.

22. The kit of claim 21 wherein the writing is on paper.

23. The kit of claim 21 wherein the writing is made available via a website.

24. The kit of claim 21 wherein the writing comprises a photograph.

25. The kit of claim 11 wherein the indication comprises software configured to detect the result as input and the classification of the subject into a cohort as output.

26. The kit of claim 25 wherein the software is incorporated into a machine configured to detect the mutant.
